(19) United States
(12) Patent Application Publication
(10) Pub. No.: US 2025/0339011 A1
(43) Pub. Date: Nov. 6, 2025

(54) SYSTEMS AND METHODS FOR MEDICAL DEVICE USAGE MONITORING

(71) Applicant: Boston Scientific Scimed, Inc., Maple Grove, MN (US)

(72) Inventors: Eric F. BROWN, Wilmington, MA (US); Louis J. BARBATO, Franklin, MA (US); James Frederick SAWICKI, Norfolk, MA (US); Mildred ANTWI-NSIAH, Brighton, MA (US); William J. ABNER, Rutland, MA (US)

(73) Assignee: Boston Scientific Scimed, Inc., Maple Grove, MN (US)

(21) Appl. No.: 19/173,358

(22) Filed: Apr. 8, 2025

Related U.S. Application Data
(60) Provisional application No. 63/642,907, filed on May 6, 2024.

(51) Int. Cl. A61B 1/00

(52) U.S. Cl. CPC A61B 1/00006; A61B 1/00045

(57) ABSTRACT

A medical device is determined not to be expired based on usage data received from a secure memory of the medical device upon connection to a computing system. Image data received from the medical device is processed and displayed. Usage metric(s) associated with the medical device are iteratively obtained and written to the secure memory, and compared to corresponding usage metric limit(s) to determine whether the medical device is expired. In response to determining expiration, an expiration value is written to the secure memory for storage. After determining expiration, the image data received is continued to be processed and displayed without interruption until the medical device is disconnected. Upon a next connection of the medical device to the computing system or another computing system, any image data received is failed to be processed and displayed based on the usage data received from the secure memory.

### CROSS-REFERENCE TO RELATED APPLICATIONS

This application claims the benefit of priority to U.S. Provisional Application No. 63/642,907, filed on May 6, 2024, which is incorporated by reference herein in its entirety.

### TECHNICAL FIELD

The disclosure relates generally to systems and methods for monitoring usage of medical devices. More specifically, aspects of the disclosure pertain to systems and methods for monitoring usage, and optionally preventing reuse, of limited use medical devices when allowable usage limits have been reached or exceeded.

### BACKGROUND

A medical system may include a medical device, such as an endoscope, that is removably connectable to a computing system to perform a medical procedure. For example, the endoscope may be inserted into and navigated through a body lumen of a patient to a target site during a medical procedure. The endoscope may include an imaging device configured to capture images, as well as a light source configured to emit light to facilitate visualization of anatomical features and/or or other objects within the lumen that are captured by the images. The computing system may process the captured images received from the endoscope and display the processed images for viewing by an operator (e.g., a physician).

Endoscopes are complex instruments that contain many small working parts. As a result, it can be challenging to fully clean and disinfect endoscopes after use in medical procedures. If an endoscope is not thoroughly cleaned and disinfected after use, tissue or fluid from one patient may remain in the endoscope. If the endoscope is then used in a subsequent medical procedure on a different patient, such failure to thoroughly clean or disinfect the endoscope can result in cross-contamination and thus patient-to-patient transmission of infection. Therefore, in the endoscopy space, endoscopes that are single use or disposable are increasingly being used to help avoid cross-contamination and prevent infection transmission between patients.

### SUMMARY

According to one aspect, the techniques described herein relate to computing systems. An example computing system includes at least one memory configured to store instructions, and at least one processor configured to execute the instructions to perform operations to monitor usage of a medical device removably connected to the computing system. The operations comprise: detecting a connection of the medical device to the computing system; determining the medical device is not expired based on usage data received from a secure memory of the medical device upon connection; and processing and causing display of image data received from an imaging device of the medical device. The operations also comprise, iteratively: obtaining one or more usage metrics associated with the medical device; writing the one or more usage metrics to the secure memory of the medical device for storage as part of the usage data; comparing the one or more usage metrics to one or more corresponding usage metric limits; and determining whether the medical device is expired based on the comparison. The operations further comprise: in response to determining the medical device is expired, writing an expiration value to the secure memory of the medical device for storage as part of the usage data; and after the medical device is determined to be expired, continuing to process and cause display of the image data received from the imaging device of the medical device without interruption until the medical device is disconnected from the computing system. Upon a next connection of the medical device to the computing system or another computing system, the computing system or the other computing system determines the medical device is expired based on the usage data received from the secure memory of the medical device upon the next connection, and fails to process and cause display of any image data received from the imaging device of the medical device.

In some examples, the secure memory of the medical device includes a data structure comprising a plurality of data fields for storing the usage data. The plurality of data fields includes one or more usage metric fields corresponding to the one or more usage metrics. Values of the one or more usage metric fields initially written to the secure memory at a time of manufacturing of the medical device are one or more of null or zero values, and writing the one or more usage metrics to the secure memory of the medical device comprises: causing the values of the one or more usage metric fields corresponding to the one or more usage metrics to be one or more of populated or incremented according to the one or more usage metrics obtained.

In other examples, the plurality of data fields further includes one or more usage metric limit fields storing one or more maximum allowed values representing the one or more corresponding usage metric limits for the one or more usage metrics. Comparing the one or more usage metrics to the one or more corresponding usage metric limits comprises: receiving the one or more maximum allowed values representing the one or more corresponding usage metric limits from the secure memory; and comparing one or more current values of the one or more usage metrics obtained to the one or more maximum allowed values. The plurality of data fields further includes an expiration data field, and writing the expiration value to the secure memory of the medical device causes the expiration data field to be updated to indicate an expired status of the medical device.

In further examples, the one or more usage metrics include one or more of: an amount of usage time, a number of connections, a date and time of a first connection of the medical device to any computing system, or a date and time of a most recent connection to the computing system. The one or more corresponding usage metric limits include a usage time limit, a connection limit, or a time since first connection limit. The computing system further comprises a real time clock, the one or more usage metrics include at least an amount of usage time, and obtaining the one or more usage metrics comprises: using the real time clock to obtain a current value for the amount of usage time from the connection of the medical device to the computing system.

In some examples, writing the one or more usage metrics to the secure memory of the medical device comprises: encrypting the one or more usage metrics transmitted to the secure memory via a data communication channel established between the computing system and the secure memory. The secure memory prevents unauthorized access to and alteration of the one or more usage metrics written to the secure memory of the medical device.

In further examples, in response to determining the medical device is expired, a notification is generated and caused to be displayed by a display device associated with the computing system. The notification indicates the medical device is expired. Prior to determining the medical device is expired based on the comparison, a loss of data communication between the computing system and the medical device is determined, the loss of data communication preventing the writing of the one or more usage metrics to the secure memory. In response to determining the loss of data communication exceeds a predefined period of time, a notification indicating the loss of data communication is generated and caused to be displayed. Based on any loss of data communication, an expired status is stored in association with an identifier of the medical device in one or more of a local or remote data store.

According to another aspect, the techniques described herein relate to methods. An example method is performed by a computing system, to which a medical device is removably connectable to, for use during a medical procedure. The method comprises: detecting a connection of the medical device to the computing system, the medical device including a secure memory storing usage data in a plurality of data fields, the plurality of data fields including one or more usage metric fields corresponding to one or more usage metrics and an expiration data field; determining the medical device is not expired based on the usage data received from the secure memory of the medical device upon connection; and processing and causing display of image data received from an imaging device of the medical device. The method also comprises, iteratively: obtaining the one or more usage metrics associated with the medical device; writing the one or more usage metrics to the secure memory of the medical device for storage as part of the usage data, the writing causing initially null or zero values of the one or more usage metric fields corresponding to the one or more usage metrics to be one or more of populated or incremented according to the one or more usage metrics obtained; comparing the one or more usage metrics to one or more corresponding usage metric limits; and determining whether the medical device is expired based on the comparison. The method further comprises: in response to determining the medical device is expired, writing an expiration value to the secure memory of the medical device for storage as part of the usage data, the writing causing the expiration data field to be updated to indicate an expired status of the medical device; and after the medical device is determined to be expired, continuing to process and cause display of the image data received from the imaging device of the medical device without interruption until the medical device is disconnected from the computing system. Upon a next connection of the medical device to the computing system or another computing system, the computing system or the other computing system determines the medical device is expired based on the usage data received from the secure memory of the medical device upon the next connection, and fails to process and cause display of any image data received from the imaging device of the medical device.

In some examples, the plurality of data fields further includes one or more usage metric limit fields storing one or more maximum allowed values representing the one or more corresponding usage metric limits, and comparing the one or more usage metrics to the one or more corresponding usage metric limits comprises: receiving the one or more maximum allowed values representing the one or more corresponding usage metric limits from the secure memory; and comparing one or more current values of the one or more usage metrics obtained to the one or more maximum allowed values.

According to a further aspect, the techniques described herein relate to computing systems. An example computing system is a computing system of a medical device that is removably connectable to a computing system for use during a medical procedure. The computing system comprises an external memory device, and a processor comprising an internal memory device and an internal timer, and configured to perform operations to monitor usage of the medical device. The operations comprise, upon a connection of the medical device to the computing system, iteratively: obtaining a usage time for the medical device from the internal timer; writing the usage time to the internal memory device; comparing the usage time to a usage time limit; and determining whether the medical device is expired based on the comparison. The operations also comprise, in response to determining the medical device is expired: writing an expiration value to the internal memory device; and generating and providing an expiration indication to the computing system, wherein the computing system continues to process and cause a display of image data captured by an image device until the medical device is disconnected from the computing system. The operations further comprise, upon a next connection of the medical device to the computing system or another computing system, and based on the expiration value written to the internal memory device, generating and providing the expiration indication to the computing system or the other computing system, wherein the computing system or the other computing system fails to process and cause a display of any image data captured by the image device based on the expiration indication.

In some examples, the usage time obtained from the internal timer is a first usage time, and the operations further comprise: receiving a second usage time obtained by the computing system; comparing the first usage time to the second usage time; and determining no discrepancy based on a difference between the first usage time and the second usage time being below a predefined threshold.

In further examples, the usage time obtained from the internal timer is a first usage metric, and the operations further comprise: receiving a second usage metric obtained by the computing system; and comparing each of the first usage metric and the second usage metric to corresponding usage metric limits, wherein determining whether the medical device is expired is based on at least one of the first usage metric and the second usage metric exceeding the corresponding usage metric limits.

It may be understood that both the foregoing general description and the following detailed description are exemplary and explanatory only and are not restrictive of the invention, as claimed. As used herein, the terms “comprises,” “comprising,” “includes,” “including,” “has,” “having,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The term “approximately,” or like terms (e.g., “substantially”), includes values+/−10% of a stated value.

### BRIEF DESCRIPTION OF THE DRAWINGS

The accompanying drawings, which are incorporated in and constitute a part of this specification, illustrate examples of this disclosure and, together with the description, serve to explain the principles of the disclosure.

FIG. 1A depicts an example medical system, including a medical device, for which a usage monitoring process may be implemented.

FIG. 1B depicts another example medical system, including another medical device, for which a usage monitoring process may be implemented.

FIG. 2 depicts an example process for monitoring usage of the medical device of FIG. 1A.

FIGS. 3A-3D depict example processes for monitoring usage of the other medical device of FIG. 1B.

FIG. 4 depicts an example computing device.

### DETAILED DESCRIPTION

As briefly mentioned above, a medical device that is removably connectable to a computing system to perform a medical procedure, such as an endoscope, may be manufactured and instructed for use as a single use or disposable device to help avoid cross-contamination and prevent infection transmission between patients. Nonetheless, certain operators may inappropriately reuse the medical device. For example, after disconnecting the medical device from the computing system at an end of a first medical procedure of a first patient, instead of being properly disposed or discarded, the medical device may be cleaned and disinfected, and connected to the computing system for reuse in a second medical procedure of a second patient. Aspects of this disclosure are directed to systems and methods for monitoring usage, and optionally enforcing reuse prevention, of single use or disposable medical devices to increase patient safety by helping to mitigate risk of cross-contamination and infection transmission.

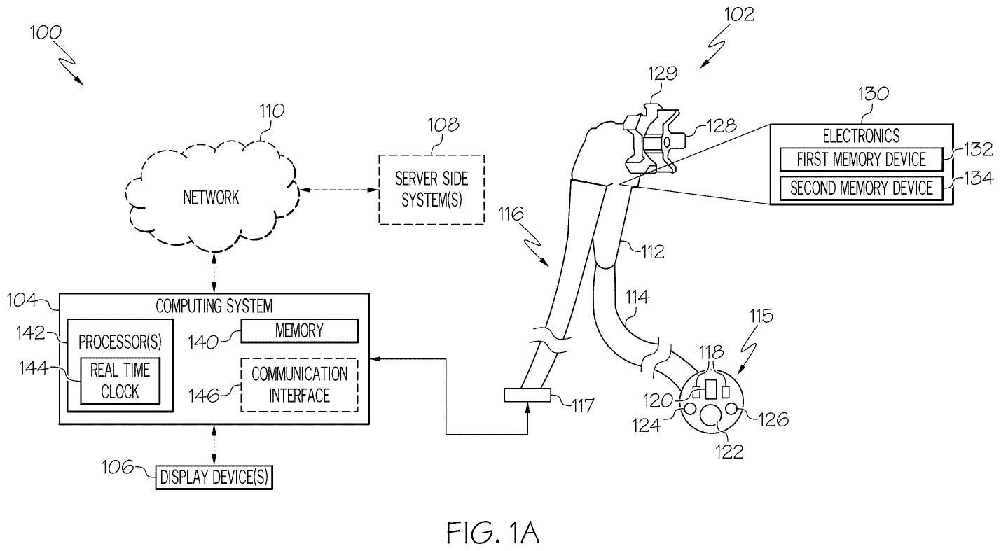

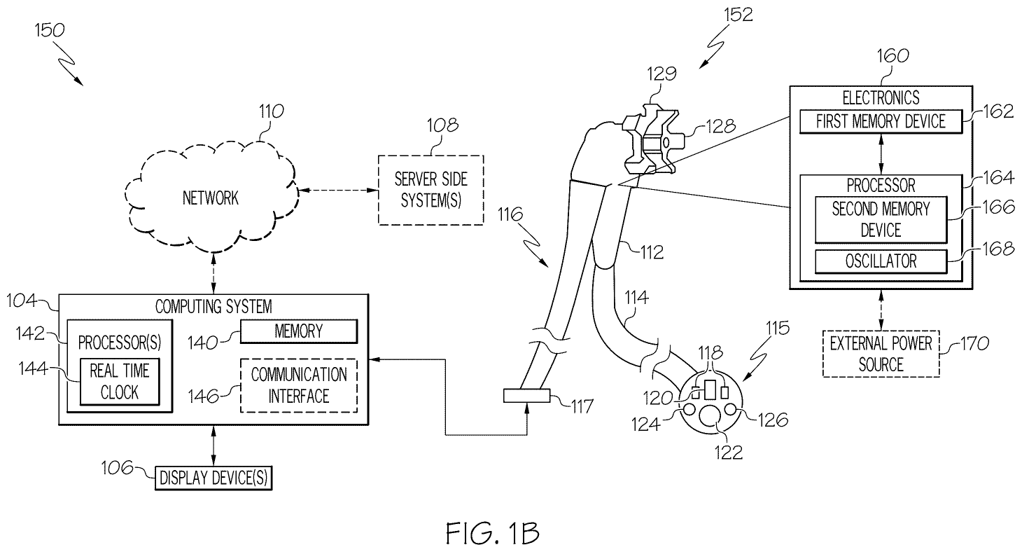

FIG. 1A depicts an exemplary medical system 100. Medical system 100 may include a medical device 102, a computing system 104, and one or more display device(s) 106. In some examples, medical system 100 may also include one or more optional server side system(s) 108 and an optional network 110 to communicatively couple at least computing system 104 to optional server side system(s) 108. In medical system 100, obtaining (e.g., tracking) of usage data associated with medical device 102 and usage monitoring based on the obtained usage data may be performed by computing system 104.

Medical device 102 may be used to perform a diagnostic and/or interventional medical procedure on a patient upon connection with computing system 104. Medical device 102 may be a single use device that is discarded or disposed of upon disconnection from computing system 104 (e.g., at an end of the medical procedure). Therefore, although only one of medical device 102 is shown in FIG. 1A, medical system 100 may include one or more other medical devices 102 that are the same or similar to medical device 102, and may also be removably connected to computing system 104 for performing medical procedures.

In some examples, and as shown in FIG. 1A, medical device 102 may be an endoscope or other type of scope or medical device having imaging capabilities, such as a cholangioscope, bronchoscope, ureteroscope, duodenoscope, gastroscope, endoscopic ultrasonography (“EUS”) scope, colonoscope, laparoscope, arthroscope, cystoscope, aspiration scope, sheath, or catheter, among other examples.

Medical device 102 may be comprised of a handle 112, a shaft 114, and an umbilicus 116. At least a portion of shaft 114, including a distal end 115 of shaft 114, may be inserted into and navigated through a body lumen of a patient to a target site during a medical procedure, such that distal end 115 is positioned proximate to the target site. Distal end 115 may include one or more illumination devices 118, an imaging device 120, and/or distal openings for one or more of a working channel 122, an insufflation or suction channel 124 and/or a fluid irrigation channel 126. Distal end 115 is shown in FIG. 1A as distal or front facing, with its components facing distally. However, in other examples, distal end 115, may be side-facing. That is, illumination devices 118, imaging device 120, and optionally one or more other distal end components, such as openings of working channel 122, suction channel 124 and/or fluid irrigation channel 126, may face radially outward, perpendicularly, approximately perpendicularly, or otherwise transverse to a longitudinal axis of the shaft 114 and distal end 115. Additionally or alternatively, distal end 115 may include one or more imaging devices 120 that face in more than one direction. For example, a first imaging device 120 may face radially outward, and a second imaging device 120 may face distally (approximately parallel to a longitudinal axis of distal end 115 and shaft 114).

Imaging device 120 may be configured to continuously capture image signals during the medical procedure as distal end 115 of medical device 102 is inserted into and navigated through the body lumen of the patient to the target site. Imaging device 120 may include one or more cameras, one or more image sensors (including analog and/or digital sensors), one or more endoscopic viewing elements, or one or more optical assemblies including one or more image sensors and one or more lenses, among other similar devices. Illumination devices 118 may be configured to receive and/or emit light to illuminate areas of the patient's body (e.g., the target site) during the medical procedure to facilitate imaging of the target site by imaging device 120. Illumination devices 118 may include one or more LEDs, incandescent light sources, optical fibers, and/or other illuminators.

A distal portion of shaft 114 that is connected to distal end 115 may have a steerable section. The steerable section may include, for example, an articulation joint. Shaft 114 and the steerable section may include a variety of structures which are known or may become known in the art. Working channel 122 may be a lumen that extends through shaft 114 and provides for delivery of instruments or treatment objects to the target site and/or removal of objects from the target site at distal end 115. Insufflation or suction channel 124 may be a lumen that extends through shaft 114 and provides insufflation or suctioning functionality at distal end 115. Fluid irrigation channel 126 may be a lumen that extends through the shaft 114 and provides irrigation functionality at distal end 115, for cleaning imaging device 120 or clearing the target site for better visualization, for example.

Handle 112 may include one or more actuators. The actuators may provide control over the steerable section, an elevator, and/or imaging functions at distal end 115 of shaft, as well as allow for the provision of air, water, suction, etc. For example, handle 112 may include control knobs 128, 129 for left, right, up, and/or down control of the steerable section of shaft 114. For example, one of control knobs 128, 129 may provide left/right control of the steerable section, and the other of control knobs 128, 129 may provide up/down control of the steerable section. Handle 112 may further include one or more locking mechanisms (e.g., knobs or levers) for preventing steering and/or braking of the steerable section in at least one of an up, down, left, or right direction. Handle 112 may include an elevator control lever (e.g., if medical device 102 is a duodenoscope or an endoscopic ultrasound scope). The elevator control lever may raise and/or lower an elevator, via a connection between the lever and an actuating wire that extends from the lever, through shaft 114, to the elevator.

Additionally, medical device 102 includes electronics 130. In some examples, and as shown in FIG. 1A, electronics 130 may be positioned within handle 112. Electronics 130 may include a first memory device 132 and a second memory device 134. First memory device 132 and second memory device 134 may each be or include an electrically erasable programmable read-only memory (EEPROM). However, first memory device 132 may be an unsecure EEPROM, while second memory device 134 may be a secure EEPROM. In other words, first memory device 132 may be an unsecure memory device and second memory device 134 may be a secure memory device.

First memory device 132 may be configured to store calibration and manufacturing data for medical device 102, among other types of data that do not require storage within a secure memory device. The calibration and manufacturing data may be written to first memory device 132 during manufacturing. Among other example uses, the calibration and manufacturing data may be communicated from medical device 102 to computing system 104 during an initialization process to facilitate a set-up of (e.g., establish) operating parameters of medical device 102 upon connection to computing system 104. Second memory device 134, being the secure memory device, may be configured to store any data type that is to be protected from external probing and/or alteration. These data types may include, but are not limited to, usage data and associated policies, a software version identifier associated with medical device 102, an identifier of medical device 102, and authentication-related data.

With respect to the usage data, second memory device 134 may store a table or other similar data structure having a plurality of data fields, including data fields associated with usage metrics to be collected for medical device 102 upon connection of medical device 102 to computing system 104 (e.g., usage metric fields). Example usage metrics collected may include an amount of time used, a number of connections, a date and time of first connection, a date and time of most recent connection, etc. Initially, the table or data structure may be written to second memory device 134 during manufacturing with the usage metric fields having null and/or zero values. As part of an example usage monitoring process, updates to the table or data structure to, for example, populate or increment values of the usage metric fields, may be written to second memory device 134 by computing system 104.

In some examples, the data fields may also include data fields representing usage metric limits for the usage metrics (e.g., usage metric limit fields). The usage metric limit fields may be populated with values (e.g., maximum allowed values) during manufacturing, and these values may be obtained and used as part of the usage monitoring process to determine whether medical device 102 has expired. Example usage metric limits may include a usage time limit (e.g., a maximum amount of time that medical device 102 can be used), a connection limit (e.g., a maximum number of connections allowed), and/or a time since first connection limit (e.g., a maximum amount of time elapsed from the first connection).

In further examples, at least one of the data fields is an expiration data field, where a presence or absence of a particular value (e.g., a bit), flag, or other indicator within this expiration data field identifies a status of medical device 102 as expired or not expired.

The software version identifier associated with medical device 102 stored in second memory device 134 may identify a current version of software (e.g., program or instructions) stored and executable by medical device 102 to enable communication with computing system 104. Because the software version identifier may be relied upon by computing system 104 to properly interpret the data stored on medical device 102, the software version identifier is stored in second memory device 132 (e.g., a secure memory device) to prevent the software version identifier from being inadvertently or maliciously modified.

The identifier of medical device 102 stored in second memory device 134 may include a serial number or other similar information for identifying medical device 102. In some examples, a portion of the identifier may indicate a general type of medical device 102, in addition to providing information unique to the particular medical device 102 of that general type. For example, the identifier may be a serial number or a universal product number (UPN).

The authentication-related data may be leveraged to determine whether an integrity of the data stored in second memory device 134 has been maintained (e.g., has not been altered or otherwise tampered with) since manufacturing, when medical device 102 is connected to computing system 104.

In some examples, data communication between computing system 104 and second memory device 134 may be encrypted to provide additional security. Additionally, second memory device 134 may include further security features to prevent unauthorized access to stored data within second memory device 134, including the usage data. Resultantly, unauthorized reading and/or alteration of the usage data is prevented.

Umbilicus 116 connects handle 112, shaft 114, distal end 115, and components at distal end 115 to one or more sources of, for example, power, imaging device control, image processing, light, light control, and/or display equipment. As one example, umbilicus 116 may also connect to computing system 104 (e.g., via a connector plug 117), and support the transmission of various data signals between computing system 104 and medical device 102. For example, umbilicus 116 may support the transmission of data, such as calibration and manufacturing data and usage data, between medical device 102 and computing system to facilitate communication establishment and set-up of operating parameters, as well as usage monitoring. Additionally, umbilicus 116 may support the transmission of power and control signals from computing system 104 to medical device 102, and particularly to imaging device 120 and illumination devices 118. Further, umbilicus 116 may support the transmission of image data from imaging device 120 to computing system 104 for processing and display via display device(s) 106.

Computing system 104 may be a controller, a control unit, a computing device, or other similar standalone processing unit separate from and removably connectable to medical device 102. Computing system 104 may include a memory 140 and one or more processor(s) 142. Memory 140 may store instructions to be executed by processor(s) 142 to cause computing system 104 to perform corresponding operations. Memory 140 may also include one or more data stores. Additionally or alternatively, computing system 104 may include one or more data stores separate from memory 140. In some examples, processor(s) 142 may be or include a field-programmable gate array (FPGA), a digital signal processing (DSP) processor, a graphics processing unit (GPU), or the like. Additionally, processor(s) 142 may include a real time clock 144 configured to, among other things, measure or track time related to usage of a device connected to computing system 104, such as medical device 102.

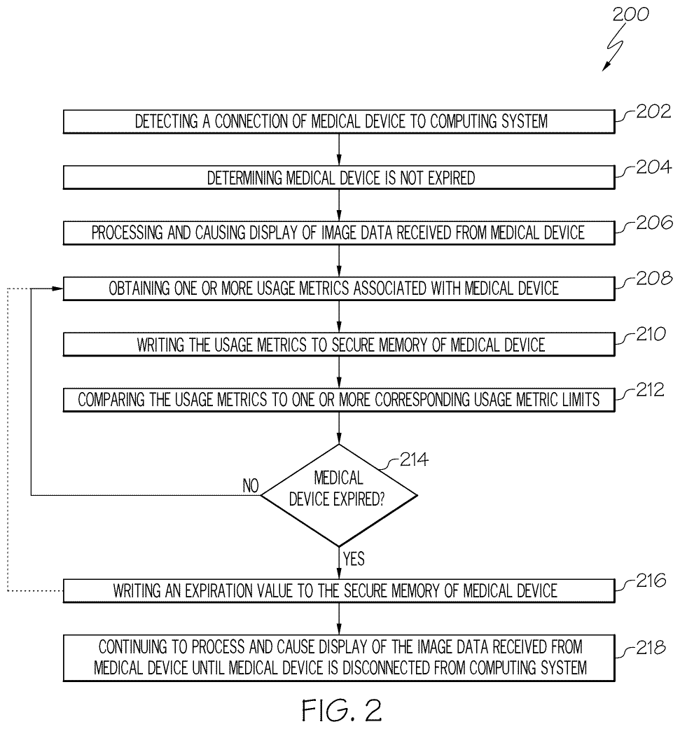

In medical system 100, where computing system 104 performs the usage monitoring process, at least a portion of the instructions stored in memory 140 may include instructions for performing the usage monitoring of medical device 102, as described in greater detail below with reference to FIG. 2. In some examples, instead of the usage metric limits being stored in second memory device 134, the instructions for the usage monitoring process may include usage metric limits (e.g., maximum allowed values for different usage metric types), where the usage metric limits may be dependent on (e.g., associated with) a type of medical device. Additionally, the instructions may include other types of limits, such as a predefined threshold period of time following a loss of communication between second memory device 134 and computing system 104 that may be leveraged as part of the usage monitoring process. At least one of processor(s) 142 may be configured to execute these instructions to perform the usage monitoring process.

In some examples, when medical device 102 has imaging capabilities, the instructions stored in memory 140 may also include one or more image processing operations. Additionally, processor(s) 142 may include at least one image processor configured to process, based on the stored instructions, image data captured by imaging components of medical device 102 and provided to computing system 104 to generate images.

Computing system 104 may further include an optional communication interface 146 for providing connectivity to optional network 110 to facilitate communication with optional server side system(s) 108. Although not shown in FIG. 1A, optional communication interface 146 may also provide connectivity to medical device 102 and/or display device(s) 106. In some examples, a communicative connection between computing system 104 and medical device 102 and/or computing system 104 and display device(s) 106 may be at least partially supported via optional network 110.

Although only one computing system 104 is shown in FIG. 1A, in some examples, medical system 100 may include one or more other computing systems 104 that are the same or similar to computing system 104 to which medical device 102 may also be removably connected to, as described below. To provide an illustrative example, within a medical facility, multiple procedural suites may each include one computing system 104 that remains located in that suite.

Display device(s) 106 may be configured to display data associated with one or more of the medical device 102 and/or computing system 104. In some examples, displayed data may include information associated with the usage of medical device 102 determined as part of the usage monitoring process. Additionally, when medical device 102 includes the imaging capabilities, the displayed data may also include images generated by computing system 104. Display device(s) 106 may include one or more a combination of monitors, computing device screens, touch screen display devices, etc. In some examples, one or more of display device(s) 106 may be a separate device from computing system 104 that is communicatively coupleable to computing system 104 via wired and/or wireless connections. In other examples, at least one of display device(s) 106 may be a display or screen of computing system 104 itself.

In some examples, computing system 104 may generate, or may cause to be generated, one or more graphical user interfaces based on instructions or information stored in memory 140, instructions or information received from one or more optional server side system(s) 108, and/or the like and may cause the graphical user interfaces to be displayed via display device(s) 106. The graphical user interfaces may include text, visual elements, controls, and/or the like, in addition to the displayed data. Display device(s) 106 may include a touch screen or a display with other input systems (e.g., a mouse, keyboard, voice, etc.) for an operator of computing system 104 to control functions of computing system 104, medical device 102 via computing system 104, and/or display device(s) 106.

One or more components of medical system 100, such as medical device 102, computing system 104, and/or display device(s) 106, may be capable of network connectivity, and may communicate with one another over a wired network or a wireless network, such as optional network 110. The network may be an electronic network. The network may include a wide area network (“WAN”), a local area network (“LAN”), personal area network (“PAN”), a cellular network (e.g., a 3G network, a 4G network, a 5G network, etc.), or the like. In other examples, the components of medical system 100 may communicate and/or connect to the network over universal serial bus (USB) or other similar local, low latency connections or direct wireless protocol. Components of medical system 100 may be connected via the network, using one or more standard communication protocols, such that the component may transmit and receive communications from each other across the network.

In some examples, when one or more of the components of medical system 100 are capable of connecting to optional network 110, such as at least computing system 104, medical system 100 may also include optional server side system(s) 108. Optional server side system(s) 108 may include one or more remote data storage systems for storing data generated by computing system 104 (e.g., image data and/or data associated with the usage monitoring process). Additionally or alternatively, when medical device 102 includes an imaging system or device, optional server side system(s) 108 may include remote image processing systems configured to perform at least a portion of the image processing, including but not limited, more resource intensive processes, such as machine learning processes (e.g., to conserve local resources of computing system 104 when network connectivity is available).

FIG. 1B depicts another exemplary medical system 150. Medical system 150 is the same as medical system 100, except for medical device 152 and optional external power source 170 removably connectable to, and configured to supply power to, medical device 152 (e.g., as a back-up power source). For example, medical device 152 replaces medical device 102. Medical device 152 may be the same as medical device 102 except for one or more electronics 160 included therein. For example, in medical system 150, usage monitoring based on one or more usage metrics obtained by medical device 152 and/or computing system 104 may be performed by medical device 152. Therefore, electronics 160 of medical device 152 include a first memory device 162 and a processor 164, including a second memory device 166 and an oscillator 168, to enable medical device 152 to perform the usage monitoring.

First memory device 162 may be an external memory device, such as an external EEPROM, that is separate from, but in communication with, processor 164. Similar to first memory device 132 of medical device 102, first memory device 162 may store calibration and manufacturing data for medical device 152. The calibration and manufacturing data may be written to first memory device 162 during manufacturing.

Processor 164 may be a microcontroller. Second memory device 166 may be an internal memory device, such as an internal EEPROM, of the microcontroller. Similar to second memory device 134 of medical device 102, second memory device 166 may be a secure memory device configured to store any data type that is to be protected from external probing and/or alteration, such as usage data and associated policies, a software version identifier associated with medical device 152, an identifier of medical device 152, and authentication-related data.

With respect to the usage data, second memory device 166 may store a table or other similar data structure having a plurality of data fields, including data fields associated with usage metrics to be collected for medical device 152 upon connection of medical device 152 to computing system 104 (e.g., usage metric fields). Example usage metrics collected may include an amount of time used, a number of connections, a date and time of first and/or most recent connection, etc. Initially, the table or data structure may be written to second memory device 166 during manufacturing with the usage metric fields having null and/or zero values. As part of an example usage monitoring process, updates to the table or data structure to, for example, populate or increment values of the usage metric fields, may be written to second memory device 166 by processor 164.

In some examples, the data fields may also include data fields representing usage metric limits for the usage metrics (e.g., usage metric limit fields). The usage metric limit fields may be populated with values (e.g., maximum allowed values) during manufacturing, and these values may be obtained and used as part of the usage monitoring process to determine whether medical device 152 has expired. Example usage metric limits may include a usage time limit (e.g., a maximum amount of time that medical device 152 can be used), a connection limit (e.g., a maximum number of connections allowed), and/or a time since first connection limit (e.g., a maximum amount of time elapsed from the first connection).

In further examples, at least one of the data fields is an expiration data field, where a presence or absence of a particular value (e.g., a bit), flag, or other indicator within this expiration data field identifies a status of medical device 152 as expired or not expired.

The software version identifier associated with medical device 152 stored in second memory device 166 may identify a current version of software (e.g., program or instructions) stored and executable by medical device 152, as described in detail below. Because the software version identifier may be relied upon by computing system 104 to properly interpret the data stored on medical device 152, the software version identifier is stored in second memory device 166 (e.g., a secure memory device) to prevent the software version identifier from being inadvertently or maliciously modified.

The identifier of medical device 152 stored in second memory device 166 may include a serial number or other similar information for identifying medical device 152. In some examples, a portion of the identifier may indicate a general type of medical device 152, in addition to providing information unique to the particular medical device 152 of that general type. For example, the identifier may be a serial number or a UPN.

The authentication-related data may be leveraged to determine whether an integrity of the data stored in second memory device 166 has been maintained (e.g., has not been altered or otherwise tampered with) since manufacturing, when medical device 152 is connected to computing system 104.

Other memory portions of processor 164 (e.g., a flash memory) may store a program or instructions for the usage monitoring process to be executed by processor 164 based, at least in part, on the usage data stored in second memory device 166. In other words, firmware including the program may be loaded onto the flash memory of processor 164. In some examples, instead of the usage metric limits being stored in the table of second memory device 166 as described above, the program or instructions for the usage monitoring process stored in the flash memory may include the usage metric limits. Additionally, the program may include other types of limits, such as a predefined threshold period of time following a loss of communication between processor 164 and computing system 104 that may be leveraged as part of usage monitoring process.

Processor 164 may also include a built-in or integrated oscillator 168 or other similar clock-like device configured to track or count time. For example, when power is supplied to electronics 160 by computing system 104 (e.g., upon connection of medical device 152 to computing system 104), oscillator 168 may be configured to begin tracking or counting time. In some examples, oscillator tics may be counted at predetermined intervals (e.g., per microsecond, per second, etc.) to track the time. The time tracked by oscillator 168 may be written to the second memory device 166 by processor 164 to update one or more of the usage metric fields in the table, such as a field related to total amount of usage time. In some examples, the writing may occur at predefined intervals. Example intervals may range from a per second interval to a per minute interval, for example. This data may then be used as part of the decisioning logic of the usage monitoring process.

Optional external power source 170 may be configured as a back-up power source to supply power to medical device 152. For example, when medical device 152 is disconnected from computing system 104 or the power supply received from computing system 104 is otherwise disrupted, processor 164 may enter into a low power state and begin to receive power from optional external power source 170. Resultantly, oscillator 168 may continue to measure or count time, and processor 164 may continue to write the time to second memory device 166. In some examples, optional external power source 170 may be a supercapacitor. In other examples, optional external power source 170 may be a battery or other similar power source.

Although various components in medical systems 100, 150 are depicted as separate components in FIGS. 1A and 1B, respectively, it should be understood that a component or portion of a component in medical systems 100, 150 may, in some embodiments, be integrated with or incorporated into one or more other components. For example, one of display device(s) 106 may be integrated with computing system 104. In some embodiments, operations or aspects of one or more of the components discussed above may be distributed amongst one or more other components. Any suitable arrangement and/or integration of the various systems and devices of medical systems 100, 150 may be used.

The specific examples included throughout the present disclosure implement an endoscopic imaging system configured to perform usage monitoring of a single use or disposable medical device having imaging capabilities. However, it should be understood that techniques according to this disclosure may be adapted to other medical systems having any type of single or limited use medical device that is connected to a computing system to enable operation thereof. It should also be understood that the examples above are illustrative only. The techniques and technologies of this disclosure may be adapted to any suitable activity.

FIG. 2 depicts an example process 200 for monitoring usage of medical device 102 of medical system 100. In some examples, one or more steps or decisions of process 200 may be performed by processor(s) 142 of computing system 104 of medical system 100.

At step 202, process 200 may include detecting a connection of medical device 102 to computing system 104. Upon connection, computing system 104 may perform a series of initialization steps to help establish data communication between computing system 104 and medical device 102, perform various checks, and/or set up operating parameters for various components of medical device 102, among other examples.

To provide an illustrative example, computing system 104 may determine the connection between medical device 102 and computing system 104 is a complete connection (e.g., performs a connection check). For example, a resistance value is measured by a detect resistor of computing system 104 that is positioned in a circuit between a net detector and a ground. If the resistance value is above a threshold value indicating a connection, the computing system 104 may cause a low amount of current to be output, as a test, to illumination devices 118 to verify medical device 102 is in complete connection (e.g., both mechanically and electrically) with computing system 104. The amount of LED current applied may be based on the resistance value detected.

Once the complete connection is verified, the resistance value may be used to configure and activate a digital power supplied from computing system 104 to medical device 102. The resistance value may also be used to determine a high-level medical device type to identify an appropriate communication protocol to enable communication with medical device 102 (e.g., device of first type or second type for communication protocol purposes). For example, if the resistance value is within a first range of values, medical device 102 may be identified as a first device type for communication protocol purposes. Similarly, if the resistance value is within a second range of values, medical device 102 may be identified as a second device type for communication protocol purposes. Example communication protocols may include the Inter-Integrated Circuit (I2C) protocol, RS-422 technical standard, and/or serial peripheral interface (SPI) standard. An appropriate communication protocol may be based on a volume of data being communicated, time constraints, data redundancy, or to limit a number of conductors needed to communication.

Computing system 104 may communicate with medical device 102 using the identified communication protocol to confirm medical device 102 is authentic. In some examples, the authentication may include an integrity check of the data stored in second memory device 134 (e.g., the secure EEPROM) to confirm second memory device 134 is secure and has not been altered and/or tampered with since manufacturing. Example authentication techniques that may be leveraged individually or in any combination with one another include, but are not limited to, hashing, unique device identification, checksum authentication, cryptographic signature application, or challenge-response interrogation. One or more of these authentication techniques may involve or use the authentication-related data stored in second memory device 134.

Once medical device 102 is determined to be authentic, computing system 104 may receive additional data from second memory device 134 of medical device 102. For example, computing system 104 may receive the usage data and associated policies stored in second memory device 134. One example policy may be associated with a reuse prevention feature for patient safety.

For example, one of the initialization steps may include determining whether or not the reuse prevention feature for patient safety is to be turned on or implemented for medical device 102 such that reuse prevention is enforced by computing system 104 based on monitored usage of medical device 102. For example, for single or limited use devices, such as medical device 102, the reuse prevention safety feature may help to ensure that medical device 102 is effectively inoperable after the single or limited use to prevent the medical device 102 from being improperly reused and causing risk of cross-contamination and/or infection transmission to patients.

In some examples, the reuse prevention determination may be made based on a value provided as part of the usage data from medical device 102 (e.g., where a value of 1 for a certain variable indicates for the feature to be on, and a value of 0 indicates for the feature to be off). In other examples, computing system 104 may utilize an identifier of medical device 102 received as part of the additional data from second memory device 134, along with the usage data, to query a lookup table or other similar data store associated with reuse prevention to determine whether the identifier of medical device 102 is a reuse prevention assigned identifier.

One or more remaining steps of process 200 may continue to be performed regardless of whether reuse prevention for patient safety is to be enforced or not. In other words, usage of medical device 102 may be monitored regardless. However, in examples, where the reuse prevention for patient safety is to be enforced, additional enforcement steps may be performed as discussed in detail below.

At step 204, process 200 may include determining medical device 102 is not expired. As previously mentioned, medical device 102 may be a single use or disposable device. Therefore, expiration may be related to whether or not medical device 102 has been previously used in a medical procedure. In some examples, the determination at step 204 is performed as one of the initialization steps upon connection (e.g., prior or in conjunction with other above-mentioned initialization steps). The determination may be based on the usage data received from medical device 102. For example, a value (e.g., a bit), flag, or other indicator associated with an expiration data field in the table stored in second memory device 134, and received by computing system 104 upon connection, may indicate that medical device 102 is not expired.

Based on medical device 102 not being expired, at step 206, process 200 may include processing and causing display of image data received from medical device 102. For example, once medical device 102 is determined to not be expired, initialization may be completed. For example, computing system 104 may receive calibration and manufacturing data stored in first memory device 132 from medical device 102. Computing system 104 may use the calibration and manufacturing data to set up operating parameters of medical device 102, including to initiate imaging device 120 and provide full power to illumination devices 118. After the initialization steps are complete, image signals captured by imaging device 120 may be received by computing system 104, and one or more image processing operations may be performed on the image signals to generate images for display on at least one of device(s) 106.

In other examples, where medical device 102 is instead determined to be expired, computing system 104 may generate and cause a notification to be displayed by at least one of device(s) 106. The notification may be a message, alert, or warning, for example, indicating expiration of medical device 102 and at least a recommendation to discontinue use thereof. In examples, where the reuse prevention for patient safety is to be enforced, the notification may be an error message, and computing system 104 may effectively render medical device 102 inoperable by, for example, failing to process and cause display of image data captured by medical device 102.

Returning to the example where medical device 102 is determined not to be expired, at step 208, process 200 may include obtaining one or more usage metrics associated with medical device 102. Example usage metrics obtained may include a connection increment (e.g., to increase a number of connections), as well as a date and time of connection, based on the detected connection of medical device 102 to computing system 104 at step 202. Additionally, the usage metrics obtained may include an amount of time that medical device 102 is being used (e.g., usage time). For example, real time clock 144 of processor(s) 142 of computing system 104 may measure or track time from connection of medical device 102 to computing system 104 until medical device 102 is disconnected from computing system 104 to represent usage time of medical device 102.

At step 210, process 200 may include writing the usage metrics to secure memory (e.g., second memory device 134) of medical device 102. For example, values in the respective usage metric fields of the table or data structure stored in second memory device 134 may be populated and/or incremented. In some examples, different types of usage metrics obtained may be written to second memory device 134 at different times. For example, the connection increment and the date and time of connection may be a first instance of usage metrics written shortly after the connection of medical device 102 to computing system 104, whereas an amount of usage time may be continuously or iteratively updated at a predetermined interval (e.g., in an interval range from about a second to a minute) throughout a duration of the connection of medical device 102 to computing system 104. In further examples, data may be written and validated in an alternating pattern to avoid data loss.

At step 212, process 200 may include comparing the usage metrics obtained at step 208 to one or more corresponding usage metric limits. In some examples, the usage metric limits may be obtained from second memory device 134. (e.g., one or more of the usage metric limit values stored at the time of manufacturing and received as part of the usage data). In other examples, the usage metric limits may be stored as part of the instructions in memory 140 of computing system 104 for performing the usage monitoring of medical device 102. The usage metric limits may include a maximum number of connections, a maximum time elapsed from a first connection of medical device 102, and/or a maximum amount of usage time, among other similar examples, that correspond to the types of usage metrics obtained by computing system 104. In some examples, the usage metric limits may also include a predefined threshold period of time associated with a loss of communication between second memory device 134 and computing system 104 that may be leveraged as part of the usage monitoring process, as described in more detail below.

In some examples, even when medical device 102 is a single use or disposable device, the maximum number of connections allowed may be more than one connection. This accounts for scenarios where, after initial connection to computing system 104 (e.g., the connection detected at step 202), medical device 102 may have to be temporarily disconnected from and reconnected to computing system 104 before or during the medical procedure. In other words, disconnection alone may not cause medical device 102 to become expired.

At decision 214, a determination of whether medical device 102 is expired is made based on the comparison of the usage metrics to the corresponding usage metric limits performed at step 212. Medical device 102 may be determined to be expired in response to at least one of the usage metrics exceeding at least one of the corresponding usage metric limits.

In response to a determination that medical device 102 is not expired at decision 214, computing system 104 may iteratively repeat steps 208-212 and decision 214 with respect to at least one of the usage metric types. For example, at least usage time may be continuously obtained and written to the secure memory of medical device 102 at the above-described predetermined interval. The comparison at step 212 and associated decision 214 may be performed on the obtained usage metrics at a same or different predetermined interval. In examples, where the same predetermined interval is used, the usage metrics may be written to the secure memory after the determination at decision 214 is made.

In response to a determination that medical device 102 is expired at decision 214, process 200 may proceed to step 216, which may include writing an expiration indication to the secure memory (e.g., second memory device 134) of medical device 102. For example, an expiration data field in the table or data structure may be updated (e.g., a bit may be flipped, a value or flag may be set, etc.) to indicate that medical device 102 is expired.

Although medical device 102 has been determined to be expired, and even if reuse prevention for patient safety is to be enforced, process 200 may optionally return to step 208 to continue to at least obtain and write usage metrics to the secure memory of medical device 102 until medical device 102 is disconnected from computing system 104. Resultantly, complete usage data logs may be generated and stored. In some examples, the usage data logs may be provided, by computing system 104, to a remote data storage system (e.g., one of optional server side system(s) 108) for storage. Such logs may be utilized to create a record or evidence regarding medical devices in circulation and the use thereof (e.g., whether it be proper or improper use).

Additionally, at step 218, process 200 may include continuing to process and cause display of the image data received from medical device 102 until medical device 102 is disconnected from computing system 104. Continuing the image processing and display helps to prevent any interruption to visualization provided to operators in a scenario where medical device 102 expires during (e.g., before a completion of) a medical procedure. In some examples, a notification indicating the expiration may be generated and displayed along with the image data to provide an alert or warning to the operators.

However, if following disconnection from computing system 104, medical device 102 is again connected to computing system 104 (or any other computing system similar to computing system 104), the medical device 102 may be determined to be expired. For example, the value (e.g., bit), flag, or other indicator associated with the expiration data field in the table stored in second memory device 134 now indicates that medical device 102 is expired (e.g., as a result of the writing at step 216). Therefore, when the usage data is received by the computing system 104 upon connection, medical device 102 may be determined to be expired. Resultantly, computing system 104 may generate and cause a notification to be displayed by at least one of device(s) 106. The notification may be a message, alert, or warning, for example, indicating expiration of medical device 102 and at least a recommendation to discontinue use thereof. In examples, where the reuse prevention for patient safety is to be enforced, the notification may be an error message, and computing system 104 may effectively render medical device 102 inoperable by, for example, failing to process and cause display of image data captured by medical device 102.

Additionally, in other aspects, if at any point during the connection of medical device 102 to computing system 104, data communication from computing system 104 to second memory device 134 is temporarily lost for a predefined period of time (e.g., meeting or exceeding the predefined threshold period of time associated with the loss of communication), the computing system 104 may generate and cause display of a communication loss notification via one of device(s) 106. The communication loss notification may include an error message, an alert, or a warning to notify the operator of the communication loss.

Due to the communication loss, usage metrics obtained by computing system 104 may not be properly written to the second memory device 134 of the medical device 102, which may affect the accuracy of the comparison performed by computing system 104 at step 210, for example. However, even if the data communication is lost, computing system 104 may continue to process and cause display of the image data received from medical device 102. In some examples, any image enhancements (e.g., performed as part of the image processing) may cease to function and/or the image data may otherwise be degraded upon continued use of medical device 102 without a reestablishment of a connection of computing system 104 to second memory device 134.

In other examples, as a result of the communication loss and associated inaccuracies with the usage monitoring, computing system 104 may store an expired status in association with the identifier (e.g., the serial number) of medical device 102 in a local data store (e.g., in memory 140) and/or a remote data store (e.g., one of the data storage systems of optional server side system(s) 108). Therefore, upon a next connection of medical device 102 to computing system 104 (or any other computing system similar to computing system 104), the medical device 102 may be determined to be expired as result of the association of the expired status with the identifier, even though the value (e.g., bit), flag, or other indicator associated with the expiration data field in the table stored in second memory device 134 may indicate otherwise.

In further aspects, if during the initialization steps, computing system 104 is unable to establish communication to the second memory device 134 of medical device 102, and thus is unable to receive the usage data, computing system 104 will not process and cause display of image data received by medical device 102.

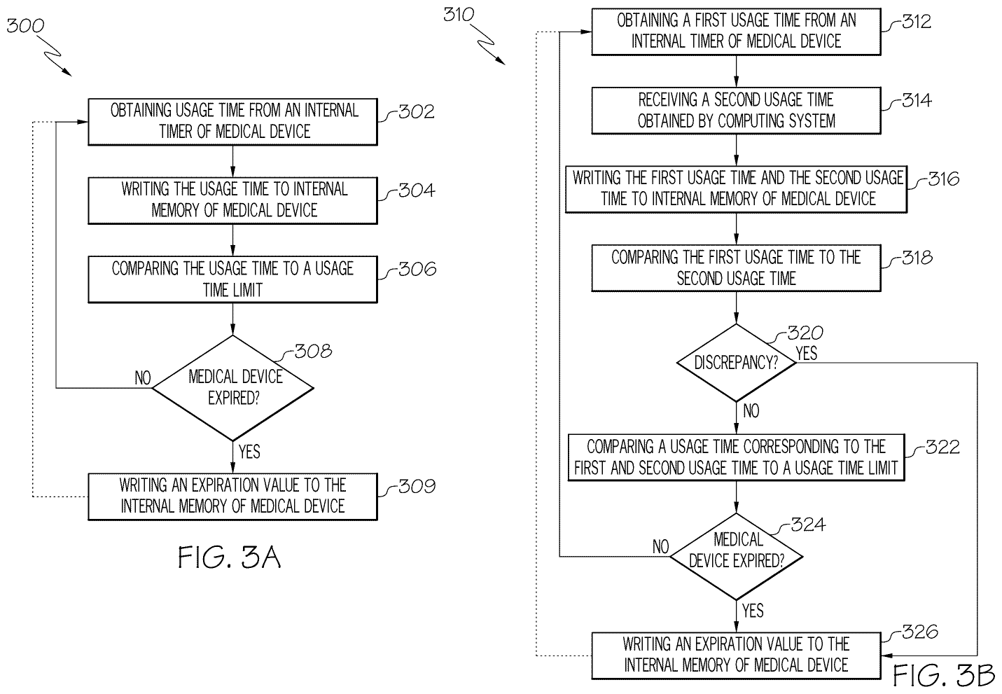

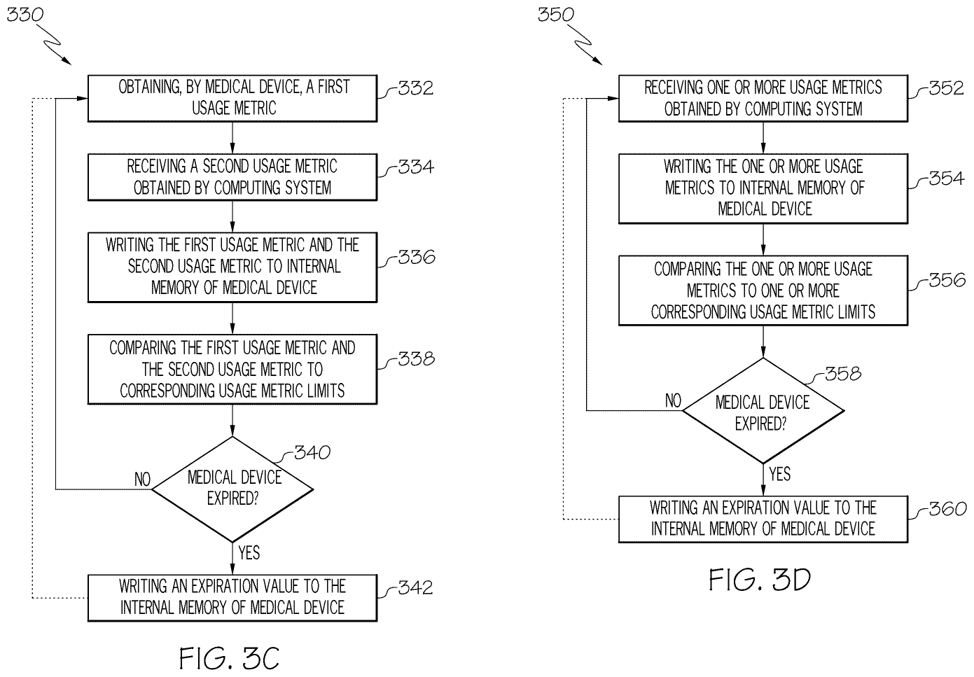

Process 200 for usage monitoring of medical device 102 has been described as being performed by processor(s) 142 of computing system 104. However, in other examples, and as described in detail with reference to FIGS. 3A-3D, usage monitoring may instead be performed by the medical device itself when such device has additional electronics (e.g., medical device 152 having electronics 160).

Process 200 described above is provided merely as an example, and may include additional, fewer, different, or differently arranged steps than depicted in FIG. 2.

FIGS. 3A-3D depict example processes 300, 310, 330, 350 for monitoring usage of medical device 152. One or more steps or decisions of each of processes 300, 310, 330, 350 may be performed by processor 164 (e.g., the microcontroller) of medical device 152.

Prior to each of processes 300, 310, 330, 350, medical device 152 may be connected to computing system 104, and a series of initialization steps may be performed by computing system 104 to help establish data communication between computing system 104 and medical device 152, perform various checks, and/or set up operating parameters for various components of medical device 152, as described in detail above with reference to step 202 of process 200. At a time of connection, a value (e.g., a bit), flag, or other indicator associated with an expiration data field in the table stored in second memory device 166 may indicate that medical device 152 is not expired. Resultantly, no expiration indication is sent to the computing system 104 as part of the initialization steps, and computing system 104 may receive, process, and cause display of image data captured by medical device 152.

Upon connection of medical device 152 to computing system 104, medical device 152 may perform usage monitoring using one or a combination of multiple of processes 300, 310, 330, 350. Each of processes 300, 310, 330, 350 vary based on type(s) of usage metric(s) obtained by the medical device 152 and/or received from computing system 104 that are used as part of the decisioning logic for the usage monitoring process.

FIG. 3A depicts example process 300 for monitoring usage of medical device 152 based on a usage metric obtained by medical device 152. At step 302, process 300 may include obtaining usage time from an internal timer, such as oscillator 168, of medical device 152. For example, upon connection to computing system 104, medical device 152 may receive power, which causes the internal timer to begin tracking or counting time. When the internal timer is oscillator 168, oscillator tics may be counted at predetermined intervals (e.g., per microsecond, per second, etc.) to track the time. Thus, the usage time may be an amount of time that medical device 152 has been connected to and powered on by computing system 104.

At step 304, process 300 may include writing the usage time to internal memory (e.g., second memory device 166) of medical device 152. For example, a value in the respective usage metric field of the table or data structure stored in second memory device 166 may be populated and/or incremented to reflect a current usage time.

At step 306, process 300 may include comparing the usage time to a usage time limit. The usage time used for the comparison may be a current value for the usage time stored in second memory device 166. Similarly, the usage time limit may be a value obtained from the second memory device 166 (e.g., one of the usage metric limit values stored at the time of manufacturing). In other examples, the usage time limit may be at least one of the usage metric limits stored, along with the program or instructions for the usage monitoring process, in the flash memory of processor 164. The usage time limit may be a maximum amount of time that medical device 152 can be used.

At decision 308, a determination of whether medical device 152 is expired is made based on the comparison of the usage time to the usage time limit performed at step 306. Medical device 152 may be determined to be expired in response to the usage time exceeding the usage time limit.

In response to a determination that medical device 152 is not expired at decision 308, process 300 may return to step 302. For example, as time passes, processor 164 may iteratively repeat steps 302-306 and decision 308. Specifically, usage time may be continuously obtained and written to the internal memory of medical device 152 at a predetermined interval (e.g., in a range from about a second to a minute between intervals). The usage time may then be compared to the usage time limit to determine whether or not medical device 152 has expired at a same or different predetermined interval.

Alternatively, in response to a determination that the medical device 152 is expired at decision 308, process 300 may proceed to step 309, which may include writing an expiration value to the internal memory (e.g., second memory device 166) of medical device 152. For example, an expiration data field in the table or data structure may be updated (e.g., a bit may be flipped, a value or flag may be set, etc.) to indicate that medical device 152 is expired. In some examples, and as discussed in more detail below, writing of the expiration indication to the internal memory may cause an expiration indication to be generated and provided to computing system 104.

In further examples, even though medical device 152 has been determined to be expired, process 300 may optionally return to step 302 and at least iteratively repeat steps 302 and 304 to continue to enable the monitoring and storage of the usage time until medical device 152 is disconnected from computing system 104.

While process 300 specifically discloses monitoring of usage time that is obtained from an internal timer of medical device 152, in other examples, alternative or additional usage metrics may be obtained or tracked by medical device 152. For example, processor 164 may be configured to identify or detect a connection based on voltage data or other similar electrical data received upon the connection of medical device 152 to computing system 104. Resultantly, the processor 164 may populate and/or increment a value in the respective usage metric field of the table or data structure stored in second memory device 166 to indicate the connection. Together with or independently from the usage time value, the populated and/or incremented value may then be compared to a connection limit (e.g., a maximum number of connections allowed) to determine whether medical device 152 is expired.

FIG. 3B depicts example process 310 for monitoring usage of medical device 152 based on a usage metric of a same type obtained by each of medical device 152 and computing system 104.

At step 312, process 310 may include obtaining a first usage time from an internal timer of medical device 152, such as oscillator 168. Step 312 may be the same or similar to step 302 described above in detail with reference to process 300. For example, the first usage time may represent of an amount of time that medical device 152 has been in use (e.g., has been connected to and powered on by computing system 104).

At step 314, process 310 may include receiving a second usage time obtained by computing system 104. For example, real time clock 144 of processor(s) 142 of computing system 104 may begin to measure or track time upon the connection of medical device 152 to computing system 104 to determine the second usage time. Computing system 104 may automatically provide the second usage time to medical device 152 at predetermined intervals and/or in response to receiving a request for the second usage time from medical device 152.

At step 316, process 310 may include writing the first usage time and the second usage time to internal memory (e.g., second memory device 166) of medical device 152. For example, values in the respective usage metric field of the table or data structure stored in second memory device 166 may be populated and/or incremented.

At step 318, process 310 may include comparing the first usage time obtained by the medical device 152 to the second usage time obtained by computing system 104. At decision 320, a determination of whether there is a discrepancy between the first usage time and the second usage time is made. In some examples, the determination may be based on a predefined threshold, such that a certain amount of discrepancy, albeit limited (e.g., within a predetermined number of seconds), is allowed. For example, if a difference between the first usage time and the second usage time is below the predefined threshold, no discrepancy may be determined. In other examples, the determination may require an exact match.

If at decision 320, no discrepancy is determined (e.g., first and second usage time match exactly and/or within the predefined threshold), process 310 may proceed to step 322. At step 322, process 310 may include comparing a usage time corresponding to the first and second usage time to a usage time limit. In examples where the first and second usage time are an exact match, the usage time for the comparison at step 322 may be a current value equal to each of the first and second usage time stored in and received from second memory device 166. In other examples where the first and second usage time are within a predefined threshold of one another, the usage time for the comparison at step 322 may be the higher value between the first and second usage time stored in and received from second memory device 166. The usage time limit may be a value obtained from the second memory device 166 (e.g., one of the usage metric limit values stored at the time of manufacturing). In other examples, the usage time limit may be at least one of the usage metric limits stored, along with the program or instructions for the usage monitoring process, in the flash memory of processor 164. The usage time limit may be a maximum amount of time that medical device 152 can be used.

At decision 324, a determination of whether medical device 152 is expired is made based on the comparison of the usage time to the usage time limit performed at step 322. Medical device 152 may be determined to be expired in response to the usage time exceeding the usage time limit.

In response to a determination that medical device 152 is not expired at decision 324, process 310 returns to step 312. For example, as time passes, processor 164 may iteratively repeat steps and/or decisions 312-324. For example, the first and second usage times may be continuously obtained and/or received and written to the internal memory of medical device 152 at one or more respective predetermined intervals (e.g., in a range from about a second to a minute between intervals). At a same or different predetermined interval, the first and second usage times may be compared to determine any discrepancy, and/or the usage time corresponding to the first and second usage times may be compared to the usage time limit to determine whether or not medical device 152 has expired.

Alternatively, in response to a determination that the medical device 152 is expired at decision 324, process 310 may proceed to step 326, which may include writing an expiration value to the internal memory (e.g., second memory device 166) of medical device 152. For example, an expiration data field in the table or data structure may be updated (e.g., a bit may be flipped, a value or flag may be set, etc.) to indicate that medical device 152 is expired. In some examples, and as discussed in more detail below, writing of the expiration indication to the internal memory may cause an expiration indication to be generated and provided to computing system 104.

Returning to decision 320, if a discrepancy is determined at decision 320, process 310 may immediately proceed from decision 320 to step 326.

In further examples, even though medical device 152 has been determined to be expired, process 310 may optionally return to step 312 and at least iteratively repeat steps 312-316 to continue to enable the monitoring and storage of the first usage time and the second usage time until medical device 152 is disconnected from computing system 104.

FIG. 3C depicts example process 330 for monitoring usage of medical device 152 based on usage metrics of different types respectively obtained by medical device 152 and computing system 104. At step 332, process 330 may include obtaining, by medical device 152, a first usage metric. In some examples, the first usage metric may be usage time obtained from an internal timer of medical device 152, such as oscillator 168. Thus, step 332 may be the same as or similar to step 302 described above in detail with reference to process 300. For example, the first usage metric may represent an amount of time that medical device 152 has been in use (e.g., has been connected to and powered on by computing system 104).

At step 334, process 330 may include receiving a second usage metric obtained by computing system 104. The second usage metric may be a different type of usage metric than the first metric. In some examples, the second metric may be a number of connections or a connection count. For example, upon connection of medical device 152 to computing system 104, computing system 104 may be configured to provide, as the second usage metric, an indication of a connection.

At step 336, process 330 may include writing the first usage metric and the second usage metric to internal memory (e.g., second memory device 166) of medical device 152. For example, values in the respective usage metric field of the table or data structure stored in second memory device 166 may be populated and/or incremented.

At step 338, process 330 may include comparing the first usage metric and the second usage metric to corresponding usage metric limits. The first usage metric for the comparison at step 338 may be a current value of usage time stored in and retrieved from second memory device 166. The second usage metric for the comparison at step 338 may be a current value for a number of connections stored in and retrieved from second memory device 166. The corresponding usage metric limits may be values obtained from the second memory device 166 (e.g., a portion of the usage metric limit values stored at the time of manufacturing). In other examples, the corresponding usage metric limits may be usage metric limits stored, along with the program or instructions for the usage monitoring process, in the flash memory of processor 164. The corresponding usage metric limits may be a usage time limit (e.g., maximum amount of time that medical device 152 can be used) and a connection limit (e.g., maximum number of connections allowed), for example.

In some examples, even when medical device 152 is a single use or disposable device, the maximum number of connections allowed may be more than one connection. This accounts for scenarios where, after initial connection to computing system 104, medical device 152 may have to be temporarily disconnected from and reconnected to computing system 104 before or during the medical procedure. In other words, disconnection alone may not cause medical device 152 to become expired.

At decision 340, a determination of whether medical device 152 is expired is made based on the comparison of first and second usage metrics to the corresponding usage metrics performed at step 322. For example, medical device 152 may be determined to be expired in response to at least one of the usage time exceeding the usage time limit or the number of connections exceeding the connection limit.

In response to a determination that medical device 152 is not expired at decision 340, process 330 may return to step 332. For example, as time passes, processor 164 may iteratively repeat steps and/or decisions 332-340. For example, the first and second usage metrics may be continuously obtained and/or received and written to the internal memory of medical device 152 at one or more respective predetermined intervals (e.g., in a range from about a second to a minute between intervals). At a same or different predetermined interval, the first and second usage metrics may be compared to the corresponding usage metric limits to determine whether or not medical device 152 has expired.

Alternatively, in response to a determination that the medical device 152 is expired at decision 340, process 330 may proceed to step 342, which may include writing an expiration value to the internal memory (e.g., second memory device 166) of medical device 152. For example, an expiration data field in the table or data structure may be updated (e.g., a bit may be flipped, a value or flag may be set, etc.) to indicate that medical device 152 is expired. In some examples, and as discussed in more detail below, writing of the expiration indication to the internal memory may cause an expiration indication to be generated and provided to computing system 104.

In further examples, even though medical device 152 has been determined to be expired, process 330 may optionally return to step 332 and iteratively repeat one or more of steps 332-336 to continue to enable monitoring and storage of the first and/or second usage metrics until medical device 152 is disconnected from computing system 104.

FIG. 3D depicts example process 350 for monitoring usage of medical device 152 based on one or more types of usage metrics obtained by computing system 104. At step 352, process 350 may include receiving one or more usage metrics obtained by computing system 104. The usage metrics may include a usage time obtained from real time clock 144 of computing system 104, a connection indication, and/or a date and a time of the connection of medical device 152 to computing system 104, for example.

At step 354, process 350 may include writing the one or more usage metrics to internal memory (e.g., second memory device 166) of medical device 152. For example, values in the respective usage metric field of the table or data structure stored in second memory device 166 may be populated and/or incremented based on the usage metrics received.

At step 356, process 350 may include comparing the one or more usage metrics to one or more corresponding usage metric limits. The usage metrics for the comparison at step 356 may be a current value of usage time, a current value for a number of connections, and/or a date and time of a first or most recent connection obtained from second memory device 166, for example, stored in and received from second memory device 166. The corresponding usage metric limits may be values stored in and received from the second memory device 166 (e.g., one of the usage metric limit values stored at the time of manufacturing). In other examples, the corresponding usage metric limits may be usage metric limits stored, along with the program or instructions for the usage monitoring process, in the flash memory of processor 164. The corresponding usage metric limits may be a usage time limit (e.g., a maximum amount of time that medical device 152 can be used), a connection limit (e.g., a maximum number of connections allowed), and/or a time since first connection limit (e.g., a maximum amount of time elapsed from the first connection), for example.

At decision 358, a determination of whether medical device 152 is expired is made based on the comparison of the one or more usage metrics to the one or more corresponding usage metrics performed at step 356. For example, medical device 152 may be determined to be expired in response to at least one of the usage metrics exceeding the corresponding usage metric limit.

In response to a determination that medical device 152 is not expired at decision 358, process 350 may return to step 352. For example, as time passes, processor 164 may iteratively repeat steps and/or decisions 352-358. For example, at least certain types of the usage metrics obtained by computing system 104 (e.g., at least usage time) may be continuously received and written to the internal memory of medical device 152 at a predetermined interval (e.g., in a range from about a second to a minute between intervals). At a same or different predetermined interval, the usage metrics may be compared to the corresponding usage metric limits to determine whether or not medical device 152 has expired.

Alternatively, in response to a determination that the medical device 152 is expired at decision 358, process 350 may proceed to step 360, which may include writing an expiration value to the internal memory (e.g., second memory device 166) of medical device 152. For example, an expiration data field in the table or data structure may be updated (e.g., a bit may be flipped, a value or flag may be set, etc.) to indicate that medical device 152 is expired. In some examples, and as discussed in more detail below, writing of the expiration value to the internal memory may cause an expiration indication to be generated and provided to computing system 104.

In further examples, even though medical device 152 has been determined to be expired, process 350 may optionally return to step 352 and iteratively repeat one or more of steps 352 and 354 to continue to enable monitoring and storage of the usage metrics until medical device 152 is disconnected from computing system 104.

Referring concurrently to FIGS. 3A-3D, regardless of process 300, 310, 330, 350 implemented, a loss of communication between second memory device 166 and computing system 104 that meets or exceeds a predefined threshold period of time may cause the processor 164 to write the expiration value to second memory device 166. For example, medical device 152 may be receiving power from and sending image data to computing system 104, but the communication channel between processor 164 and computing system 104 may be nonfunctional. If the communication channel remains nonfunctional for a period of time that meets or exceeds the predefined threshold period of time, processor 164 may write the expiration value to second memory device 166. Medical device 152 may continue to send image data to computing system 104, and computing system 104 may continue to process and cause display of the image data until medical device 152 is disconnected. In some examples, any image enhancements (e.g., performed as part of the image processing by computing system 104) may cease to function and/or the image data may otherwise be degraded upon continued use of medical device 152 without a reestablishment of communication between second memory device 166 and computing system 104.

Also, as described in detail with reference to FIG. 1B, optional external power source 170 may be a back-up power source configured to supply power to medical device 152 when medical device 152 is disconnected from computing system 104 or the power supply received from computing system 104 is otherwise disrupted. For example, processor 164 may enter into a low power state and begin to receive power from optional external power source 170. Resultantly, oscillator 168 may continue to measure or count time, and processor 164 may continue to write the time to second memory device 166 and perform the comparison to determine whether or not medical device 152 is expired. Therefore, even if medical device 152 has to be temporarily disconnected from computing system 104 during the procedure or the power supply from computing system 104 is temporarily disrupted, the disconnection or power interruption alone does not prevent medical device 152 from continued use once re-connected or power is restored.

Additionally, as briefly mentioned above, in each of processes 300, 310, 330, 350, when medical device 152 has been determined to be expired and the expiration value has been written to the internal memory, an expiration indication may be generated and provided to computing system 104. Upon receipt of the expiration indication, computing system 104 may generate a message, alert, or warning, for example, indicating the expiration of medical device 152 for display via one of device(s) 106. In some examples, computing system 104 may also associate the identifier of medical device 152 received during the initialization steps with an expired status and store the association locally and/or remotely.

Further, although medical device 152 has been determined to be expired and even if reuse prevention for patient safety is to be enforced, upon receipt of the expiration indication, computing system 104 may continue to process and cause display of the image data received from medical device 152 until medical device 152 is disconnected from computing system 104. Continuing the image processing and display helps to prevent any interruption to visualization provided to operators in a scenario where medical device 152 expires during (e.g., before a completion of) a medical procedure.

However, if following disconnection from computing system 104, medical device 152 is again connected to computing system 104 (or any other computing system similar to computing system 104), the medical device 152 may be determined to be expired. For example, the value (e.g., bit), flag, or other indicator associated with the expiration data field in the table stored in second memory device 166 now indicates that medical device 152 is expired (e.g., as a result of the writing at steps 309, 326, 342, 360 of processes 300, 310, 330, 350). Therefore, when the usage data is received by the computing system 104 upon connection, medical device 152 may be determined to be expired. Resultantly, computing system 104 may generate and cause a notification to be displayed by at least one of device(s) 106. The notification may be a message, alert, or warning, for example, indicating expiration of medical device 152 and at least a recommendation to discontinue use thereof. In examples where the reuse prevention for patient safety is to be enforced, the notification may be an error message, and computing system 104 may effectively render medical device 152 inoperable by, for example, failing to process and cause display of image data captured by medical device 152.

Each of processes 300, 310, 330, 350 described above is provided merely as an example, and may include additional, fewer, different, or differently arranged steps than depicted in FIGS. 3A-3D.

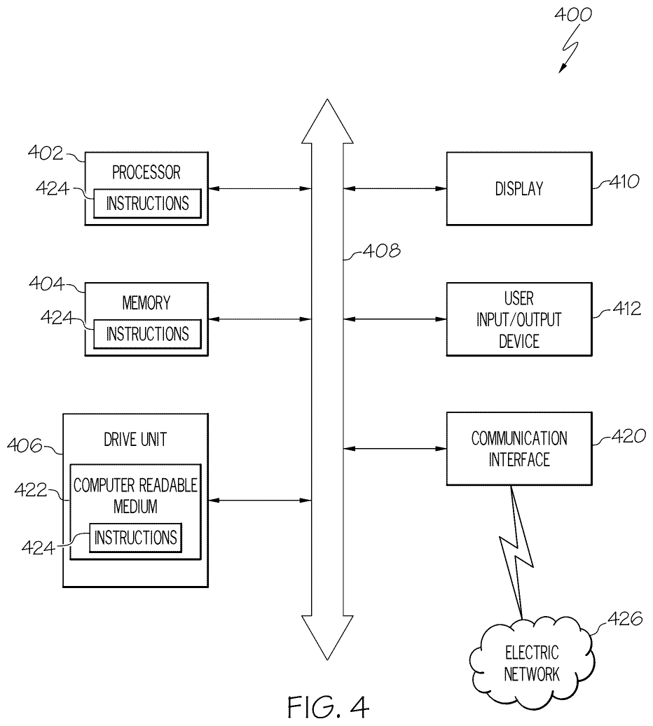

FIG. 4 depicts an example of a computer 400. FIG. 4 is a simplified functional block diagram of computer 400 that may be configured as a device for executing processes, steps, or operations depicted in, or described with respect to, FIGS. 2-3D and, according to exemplary embodiments of the present disclosure. For example, computer 400 may be configured as one or more of medical device 102, medical device 152, computing system 104, display device(s) 106, optional server side system(s) 108, and/or another device or component according to exemplary embodiments of this disclosure. In various embodiments, any of the systems herein may be or include computer 400 including, e.g., a data communication interface 420 for packet data communication. Computer 400 may communicate with one or more other computers, for example, using an electronic network 426 (e.g., via data communication interface 420). Electronic network 426 may include a wired or wireless network, for example, similar to optional network 110 depicted in FIGS. 1A and 1B.

Computer 400 also may include a central processing unit (“CPU”), in the form of one or more processors 402, for executing program instructions 424. In some examples, processors 402 may be or include one or more field-programmable gate arrays (FPGAs). Program instructions 424 may include at least instructions for performing usage monitoring (e.g., if computer 400 is computing system 104).

Computer 400 may include an internal communication bus 408. Computer 400 may also include a drive unit 406 (such as read-only memory (ROM), hard disk drive (HDD), solid-state disk drive (SDD), etc.) that may store data on a computer readable medium 422 (e.g., a non-transitory computer readable medium), although computer 400 may receive programming and data via network communications. Computer 400 may also have a memory 404 (such as random-access memory (RAM)) storing instructions 424 for executing techniques presented herein. It is noted, however, that in some aspects, instructions 424 may be stored temporarily or permanently within other modules of computer 400 (e.g., processor 402 and/or computer readable medium 422). Computer 400 also may include user input and output devices 412 and/or a display 410 to connect with input and/or output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may, at times, be communicated through the Internet or various other telecommunication networks. Such communications, e.g., may enable loading of the software from one computer or processor into another. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A computing system, comprising:
at least one memory configured to store instructions; and
at least one processor configured to execute the instructions to perform operations to monitor usage of a medical device removably connected to the computing system, the operations comprising:
detecting a connection of the medical device to the computing system;
determining the medical device is not expired based on usage data received from a secure memory of the medical device upon connection;
processing and causing display of image data received from an imaging device of the medical device;
iteratively:
obtaining one or more usage metrics associated with the medical device;
writing the one or more usage metrics to the secure memory of the medical device for storage as part of the usage data;
comparing the one or more usage metrics to one or more corresponding usage metric limits; and
determining whether the medical device is expired based on the comparison;
in response to determining the medical device is expired, writing an expiration value to the secure memory of the medical device for storage as part of the usage data; and
after the medical device is determined to be expired, continuing to process and cause display of the image data received from the imaging device of the medical device without interruption until the medical device is disconnected from the computing system,
wherein, upon a next connection of the medical device to the computing system or another computing system, the computing system or the other computing system determines the medical device is expired based on the usage data received from the secure memory of the medical device upon the next connection, and fails to process and cause display of any image data received from the imaging device of the medical device.

2. The computing system of claim 1, wherein the secure memory of the medical device includes a data structure comprising a plurality of data fields for storing the usage data.

3. The computing system of claim 2, wherein the plurality of data fields includes one or more usage metric fields corresponding to the one or more usage metrics, wherein values of the one or more usage metric fields initially written to the secure memory at a time of manufacturing of the medical device are one or more of null or zero values, and wherein writing the one or more usage metrics to the secure memory of the medical device comprises:
causing the values of the one or more usage metric fields corresponding to the one or more usage metrics to be one or more of populated or incremented according to the one or more usage metrics obtained.

4. The computing system of claim 2, wherein the plurality of data fields further includes one or more usage metric limit fields storing one or more maximum allowed values representing the one or more corresponding usage metric limits for the one or more usage metrics.

5. The computing system of claim 4, wherein comparing the one or more usage metrics to the one or more corresponding usage metric limits comprises:
receiving the one or more maximum allowed values representing the one or more corresponding usage metric limits from the secure memory; and
comparing one or more current values of the one or more usage metrics obtained to the one or more maximum allowed values.

6. The computing system of claim 2, wherein the plurality of data fields further includes an expiration data field, and wherein writing the expiration value to the secure memory of the medical device causes the expiration data field to be updated to indicate an expired status of the medical device.

7. The computing system of claim 1, wherein the one or more usage metrics include one or more of: an amount of usage time, a number of connections, a date and time of a first connection of the medical device to any computing system, or a date and time of a most recent connection to the computing system.

8. The computing system of claim 7, wherein the one or more corresponding usage metric limits include a usage time limit, a connection limit, or a time since first connection limit.

9. The computing system of claim 1, wherein the computing system further comprises a real time clock, the one or more usage metrics include at least an amount of usage time, and obtaining the one or more usage metrics comprises:
using the real time clock to obtain a current value for the amount of usage time from the connection of the medical device to the computing system.

10. The computing system of claim 1, wherein writing the one or more usage metrics to the secure memory of the medical device comprises:
encrypting the one or more usage metrics transmitted to the secure memory via a data communication channel established between the computing system and the secure memory.

11. The computing system of claim 1, wherein the secure memory prevents unauthorized access to and alteration of the one or more usage metrics written to the secure memory of the medical device.

12. The computing system of claim 1, further comprising:
in response to determining the medical device is expired, generating and causing a notification to be displayed by a display device associated with the computing system, wherein the notification indicates the medical device is expired.

13. The computing system of claim 1, further comprising:
prior to determining the medical device is expired based on the comparison, determining a loss of data communication between the computing system and the medical device, the loss of data communication preventing the writing of the one or more usage metrics to the secure memory.

14. The computing system of claim 13, further comprising:
in response to determining the loss of data communication exceeds a predefined period of time, generating and causing display of a notification indicating the loss of data communication.

15. The computing system of claim 13, further comprising:
based on any loss of data communication, storing an expired status in association with an identifier of the medical device in one or more of a local or remote data store.

16. A method performed by a computing system, to which a medical device is removably connectable to, for use during a medical procedure, the method comprising:
detecting a connection of the medical device to the computing system, the medical device including a secure memory storing usage data in a plurality of data fields, the plurality of data fields including one or more usage metric fields corresponding to one or more usage metrics and an expiration data field;
determining the medical device is not expired based on the usage data received from the secure memory of the medical device upon connection;
processing and causing display of image data received from an imaging device of the medical device;
iteratively:
obtaining the one or more usage metrics associated with the medical device;
writing the one or more usage metrics to the secure memory of the medical device for storage as part of the usage data, the writing causing initially null or zero values of the one or more usage metric fields corresponding to the one or more usage metrics to be one or more of populated or incremented according to the one or more usage metrics obtained;
comparing the one or more usage metrics to one or more corresponding usage metric limits; and
determining whether the medical device is expired based on the comparison;
in response to determining the medical device is expired, writing an expiration value to the secure memory of the medical device for storage as part of the usage data, the writing causing the expiration data field to be updated to indicate an expired status of the medical device; and
after the medical device is determined to be expired, continuing to process and cause display of the image data received from the imaging device of the medical device without interruption until the medical device is disconnected from the computing system,
wherein, upon a next connection of the medical device to the computing system or another computing system, the computing system or the other computing system determines the medical device is expired based on the usage data received from the secure memory of the medical device upon the next connection, and fails to process and cause display of any image data received from the imaging device of the medical device.

17. The method of claim 16, wherein the plurality of data fields further includes one or more usage metric limit fields storing one or more maximum allowed values representing the one or more corresponding usage metric limits, and wherein comparing the one or more usage metrics to the one or more corresponding usage metric limits comprises:
receiving the one or more maximum allowed values representing the one or more corresponding usage metric limits from the secure memory; and
comparing one or more current values of the one or more usage metrics obtained to the one or more maximum allowed values.

18. A computing system of a medical device that is removably connectable to a computing system for use during a medical procedure, the computing system comprising:
an external memory device; and
a processor comprising an internal memory device and an internal timer, and configured to perform operations to monitor usage of the medical device, the operations comprising:
upon a connection of the medical device to the computing system,
iteratively:
obtaining a usage time for the medical device from the internal timer;
writing the usage time to the internal memory device;
comparing the usage time to a usage time limit; and
determining whether the medical device is expired based on the comparison;
in response to determining the medical device is expired:
writing an expiration value to the internal memory device; and
generating and providing an expiration indication to the computing system, wherein the computing system continues to process and cause a display of image data captured by an image device until the medical device is disconnected from the computing system; and
upon a next connection of the medical device to the computing system or another computing system, and based on the expiration value written to the internal memory device, generating and providing the expiration indication to the computing system or the other computing system, wherein the computing system or the other computing system fails to process and cause a display of any image data captured by the image device based on the expiration indication.

19. The computing system of claim 18, wherein the usage time obtained from the internal timer is a first usage time, and the operations further comprising:
receiving a second usage time obtained by the computing system;
comparing the first usage time to the second usage time; and
determining no discrepancy based on a difference between the first usage time and the second usage time being below a predefined threshold.

20. The computing system of claim 18, wherein the usage time obtained from the internal timer is a first usage metric, and the operations further comprising:
receiving a second usage metric obtained by the computing system; and
comparing each of the first usage metric and the second usage metric to corresponding usage metric limits, wherein determining whether the medical device is expired is based on at least one of the first usage metric and the second usage metric exceeding the corresponding usage metric limits.
